(19) United States
(12) Patent Application Publication
(10) Pub. No.: US 2009/0076466 A1
(43) Pub. Date: Mar. 19, 2009

(54) Sutureless venous access port

(76) Inventors: Brian B. Quebbemann, Newport Beach, CA (US); Clark B. Foster, Mission Viejo, CA (US)

Correspondence Address: Morland C. Fischer, Suite 1300, 2030 Main Street, Irvine, CA, 92614, US

(21) Appl. No.: 11/901,157

(22) Filed: Sep. 17, 2007

(51) Int. Cl. A61M 39/04; A61M 29/00; A61M 39/02

(52) U.S. Cl. 604/288.02; 604/288.01; 606/190

(57) ABSTRACT

Disclosed is a compact, low profile venous access port to be implanted below the skin of a patient and coupled to a (e.g., subclavian) vein by a Seldinger technique, or the like, so that a supply of fluid medication can be delivered directly to the patient's circulatory system via a medication delivery lumen. The target axis of the venous access port herein disclosed advantageously makes a small acute angle with to the patient's skin (as opposed to the 90 degree angle that is common to conventional subcutaneous ports) so as to avoid puncturing a medication delivery lumen as a consequence of a misalignment of a needle cannula with the target area of the port. A pocket forming tool is also disclosed to be inserted through an incision made in the patient's skin to create an accurately sized anti-migration pocket that is closed around the venous access port to prevent a displacement thereof. Following implantation, anti-migration barbs which project from the venous access port attach to the patient's superficial muscular facia to hold the port in place without the use of sutures. Accordingly, the venous access port of this invention may be ideally implanted (by a radiologist) on an outpatient basis without the cost and recovery time often associated with a surgical procedure performed by a surgeon in an operating theater.

### BACKGROUND OF THE INVENTION

1. Field of the Invention

This invention relates to a low profile venous access port that is implanted below the skin and held in place without the use of sutures or the services of a surgeon so that a supply of fluid medication can be delivered through the port (by way of a needle cannula) to the circulatory system of a patient. Also disclosed is a pocket-forming tool that is adapted to form an anti-migration pocket between layers of the patient's skin in which to anchor the venous access port to be placed in fluid communication with a (e.g., subclavian) vein of the patient.

2. Background Art

Subcutaneous venous access ports are known which are infused with a liquid, whereby an ongoing regimen of medication can be delivered into the circulatory system of a patient. Such a venous access port is typically coupled to the subclavian vein by a surgeon who employs a well-known Seldinger procedure. Briefly, the surgeon first makes a relatively large incision through the skin below the clavicle and adjacent the vein. The patient's skin is then lifted up and a needle is pushed through the tissue and muscle to puncture the vein. A very narrow guide wire is fed through the needle and into communication with the vein, at which time the needle is slid off the guide wire and removed. Next, a sleeve is moved down the guide wire to widen (i.e., dilate) the puncture in the vein to be able to receive the fluid tubing that extends from the access port. The guide wire is now removed. The surgeon manipulates the patient's skin to fashion a pocket into which the access port is tucked. Finally, the access port is carefully sutured in place against the patient's tissue at the bottom of the pocket, and the original incision is closed by means of absorbable sutures. The access port is now ready to receive a cannula so that a supply of fluid medication can be injected into the patient's vein by way of the port and the fluid tubing extending therefrom to the vein.

A conventional venous access port that is often implanted and coupled to a vein of a patient by the aforementioned Seldinger technique is that commonly referred as the PORT-O-CATH fluid port. Such a fluid port is characteristically large, bulky and palpable through the patient's skin to create a wide target area for the cannula. Consequently, the fluid port may protrude through the patient's chest leading to patient discomfort and a self-conscious cosmetic appearance. In the case of this conventional fluid port, the cannula target area of the port is typically aligned perpendicular to the patient's skin. Should the incoming cannula miss the intended target area, it may accidentally strike and rupture the fluid medication carrying tubing which leads to the vein.

What is even more, because of the necessity of suturing the conventional fluid port in place (to avoid migration or rotation) and subsequently closing the relatively large incision following implantation, the services of a surgeon are commonly required. Use of a suitable operating room is likewise required. The requirements for a surgeon and an operating room facility increase the cost of the procedure. What is even more, as a consequence of the relatively large incision that is initially made to accommodate the relatively large fluid port, the patient is often subjected to increased tissue trauma, a longer recovery time, and a large and sometimes unsightly scar.

Accordingly, what would be desirable is an improved low profile, small size venous access port that can be implanted below the skin and anchored to the tissue of a patient without the need for suturing and without requiring the services of a surgeon and access to a formal operating theater so as to reduce the high costs that are usually associated with a conventional fluid access port. At the same time, the access port should require a relatively small incision and protrude less following implantation so as to avoid patient discomfort and self-consciousness.

### SUMMARY OF THE INVENTION

In general terms, a low profile, sutureless venous access port of relatively small size is disclosed to be attached to the patient's superficial muscular facia so that a supply of fluid medication can be delivered on an ongoing basis directly to the (e.g., subclavian) vein. By virtue of its small size and the avoidance of a traditional suture attachment, the venous access port of this invention can be implanted without the services of a surgeon and the necessity of an operating room. Moreover, only a relatively small incision through the patient's skin is necessary to implant the low profile access port, whereby tissue trauma and patient discomfort will be minimized, while the likelihood of bulging from the patient's chest will be reduced. Accordingly, the venous access port of this invention may be implanted in a radiologist's lab, or the like, during an outpatient procedure and without the usual expense associated with the customary surgical procedure that has heretofore been required to implant conventional fluid access ports.

The sutureless venous access port herein disclosed includes a flat foundation plate and a hollow tapered port body standing upwardly therefrom. Located within the port body is a hollow needle puncture-resistant cone. A self-sealing plug is located at the wide fluid inlet end of the puncture-resistant cone, and a spherical needle stop is located at the opposite narrow fluid outlet end thereof. A hollow lumen barb projects from the narrow end of the puncture-resistant cone. One end of a medication delivery lumen is attached in surrounding engagement with the lumen barb so as to be coupled to the fluid outlet end of the hollow tapered port body in fluid communication with the hollow interior of the puncture-resistant cone. The opposite end of the medication delivery lumen can be attached to the patient's vein by means of the well-know Seldinger procedure.

The venous access port is infused with a fluid medication by way of a needle cannula that penetrates the self-sealing plug for receipt at the hollow interior of the needle puncture-resistant cone. By virtue of the tapered body of the access port which surrounds the puncture-resistant cone, a needle axis is established towards the self-sealing plug which forms an acute angle of about 30 degrees with the patient's skin or the horizontal plane of the foundation plate. In this manner, an inadvertent needle puncture and a possible rupture of the medication delivery lumen can be avoided should an incoming needle cannula be out of alignment with the preferred needle axis and miss the targeted self-sealing plug. Each of the fluid outlet end of the port body and the narrow end of the puncture-resistant cone is deformed to have a non-round configuration. Therefore, a fluid channel is established past the opposite sides of the spherical needle stop that is disposed against the narrow deformed end of the cone. Accordingly, a continuous fluid path is created through the venous access port to the vein of the patient including the hollow interior of the puncture-resistant cone, the fluid channel past the spherical needle stop, and the medication delivery lumen that is connected between the patient's vein and the lumen barb that projects from the narrow end of the cone.

### BRIEF DESCRIPTION OF THE DRAWINGS

FIG. 1 shows a perspective view of a sutureless venous access port according to a preferred embodiment of this invention;

FIG. 2 is an exploded view of the venous access port shown in FIG. 1;

FIG. 3 is a side view of the venous access port shown in FIG. 1;

FIG. 4 is a bottom view of the venous access port showing a pair of anti-migration barbs which avoid the need for suturing the access port in place;

FIG. 5A is a cross-section of the venous access port illustrating a needle axis along which a needle cannula travels to enable the port to be infused with a fluid medication to be delivered to the vein of a patient;

FIG. 5B is a cross-section taken along lines 5B-5B of FIG. 4;

FIG. 5C is an enlarged cross-section to illustrate the position of a spherical needle stop relative to a narrow deformed end of a needle puncture-resistant cone and a hollow lumen barb projecting outwardly therefrom;

FIG. 6 is a perspective view of a pocket-forming tool to be used to create an anti-migration pocket between layers of skin of a patient within which to receive the sutureless venous access port of FIGS. 1-5;

FIG. 7 shows the venous access port sliding into receipt by the anti-migration pocket formed by the pocket-forming tool of FIG. 6;

FIG. 8 shows an incision in the patient's skin being held closed after the venous access port of FIG. 7 moves therethrough for receipt by the anti-migration pocket; and

FIG. 9 shows the anti-migration pocket closed around the venous access port after the incision of FIG. 8 has healed such that the port can be infused with a supply of fluid medication to be delivered to the vein of a patient by way of an incoming needle cannula.

### DESCRIPTION OF THE PREFERRED EMBODIMENT

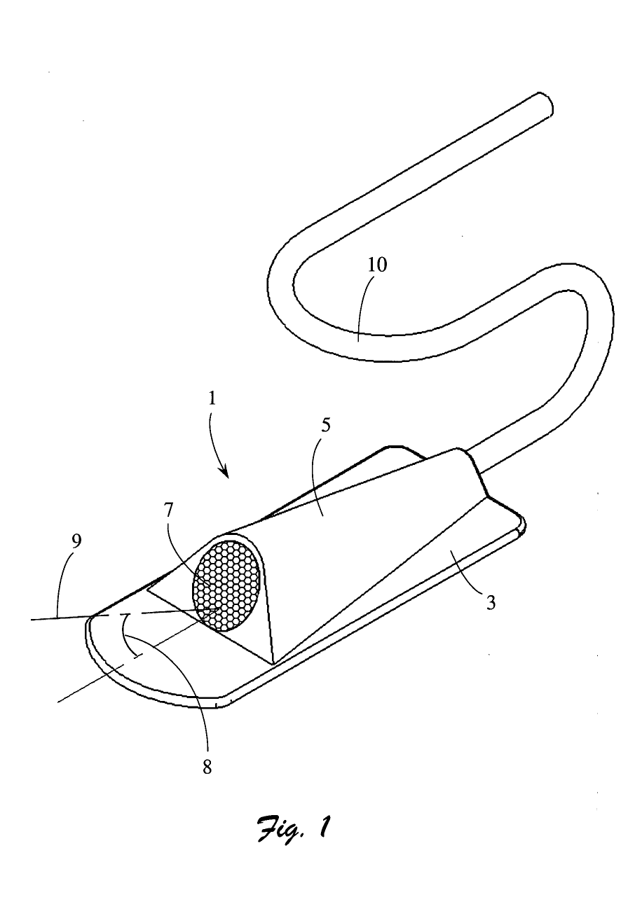

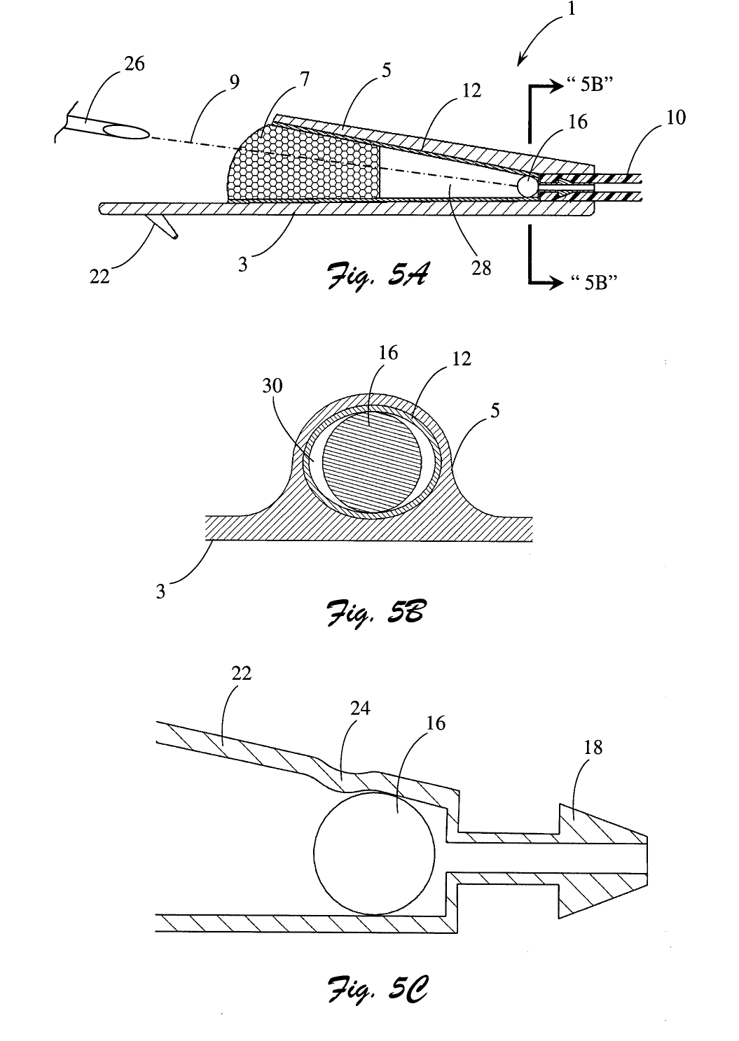

A preferred embodiment for a sutureless venous access port 1 which forms the present invention is initially described according to a preferred embodiment while referring to FIG. 1 of the drawings. Venous access port 1 includes a flat (i.e., horizontal) foundation plate 3 that is preferably manufactured from a biocompatible metal or a medical-grade plastic. A hollow tapered port body 5 having opposite proximal and distal ends is seated upon and affixed (e.g., fused) to foundation plate 3. Located inside the proximal (fluid inlet) end of the hollow body 5 is a self-sealing gelatinous plug 7 that is adapted to automatically close a puncture wound of the type produced when a needle cannula (designated 26 in FIG. 5A) passes therethrough.

The proximal end of the tapered hollow body 5 of access port 1 is sloped rearwardly so as to establish a corresponding needle target axis 9 which forms an acute angle 8 with the horizontal plane of foundation plate 3. The needle axis 9 makes an angle which is less than 90 degrees and preferably any acute angle lying between 20 and 45 degrees above the patient's skin or the horizontal plane of foundation plate 3. By virtue of the foregoing and unlike the conventional PORT-O-CATH fluid port, the profile and size (i.e., height) of access port 1 can be minimized. In the example shown in FIG. 1, the ideal profile minimizing angle 8 between the horizontal plane of foundation plate 3 and the needle axis 9 is approximately 30 degrees. Having a reduced profile is advantageous to reduce discomfort and a potential chest bulge when the venous access port 1 of this invention is implanted below the skin of a patient in need of fluid medication.

To this end, a medication delivery lumen 10 is coupled to the distal (fluid outlet) end of the hollow body 5 of access port 1. The medication delivery lumen 10 enables fluid medication to be supplied on an ongoing basis to the circulatory system of the patient within whom the venous access port 1 has been implanted by way of a needle cannula that is moved into fluid communication with port 1 by penetrating the self-sealing plug 7 along the needle axis 9 (best shown in FIG. 5A).

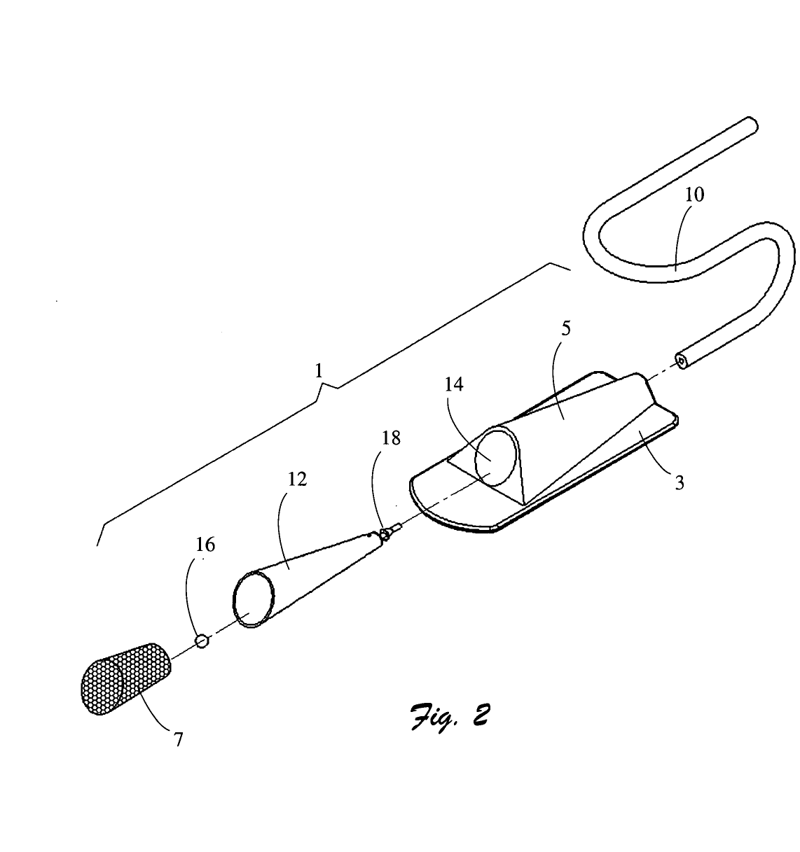

FIG. 2 illustrates an exploded view of the sutureless venous access port 1 of FIG. 1. A hollow needle puncture-resistant (e.g., metal) cone 12 is located inwardly of the hollow tapered body 5 of access port 1 by way of an opening 14 at the proximal end of body 5. The puncture-resistant cone 12 is sized to surround and retain the self-sealing plug 7 at a relatively wide end thereof. A spherical needle stop 16 is located inwardly of the opening 14 to lie at the opposite narrow end of the puncture-resistant cone 12 (best shown in FIG. 5C). The puncture-resistant cone 12 prevents an incoming needle cannula that is misaligned with respect to the preferred needle axis (designated 9 in FIG. 1) from penetrating and damaging the hollow port body 5. The needle stop 16 prevents an incoming needle cannula from moving completely through the puncture-resistant cone 12 and outwardly past the distal end of port body 5 at which to possibly penetrate and rupture the medication delivery lumen 10 that is coupled to the distal end of body 5.

In this regard, a hollow lumen barb 18 projects from the narrow end of the puncture-resistant cone 12. In the assembled configuration (best shown in FIG. 5A), one end of the medication delivery lumen 10 is moved into surrounding engagement with the lumen barb 18 of puncture-resistant cone 12 within the distal end of the hollow body 5 of venous access port 1, whereby the lumen 10 is coupled to the distal end of port body 5 in fluid communication with the puncture-resistant cone 12 located therewithin.

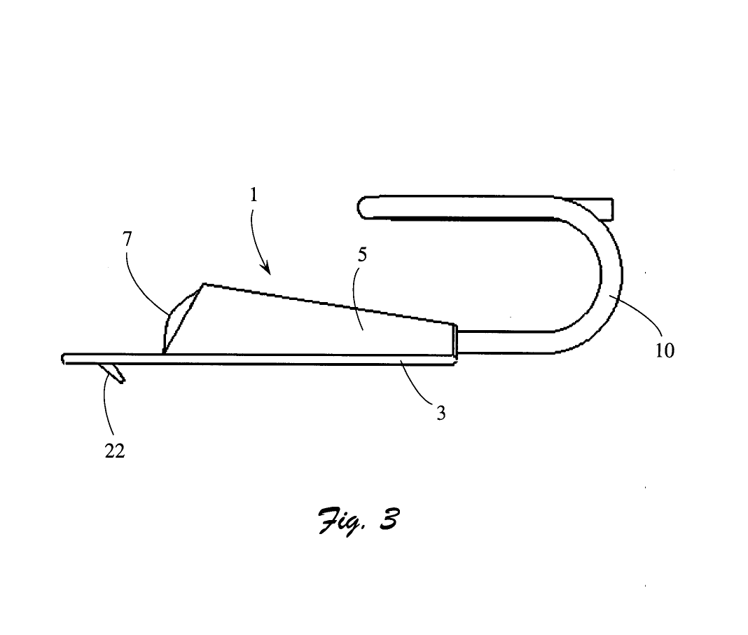

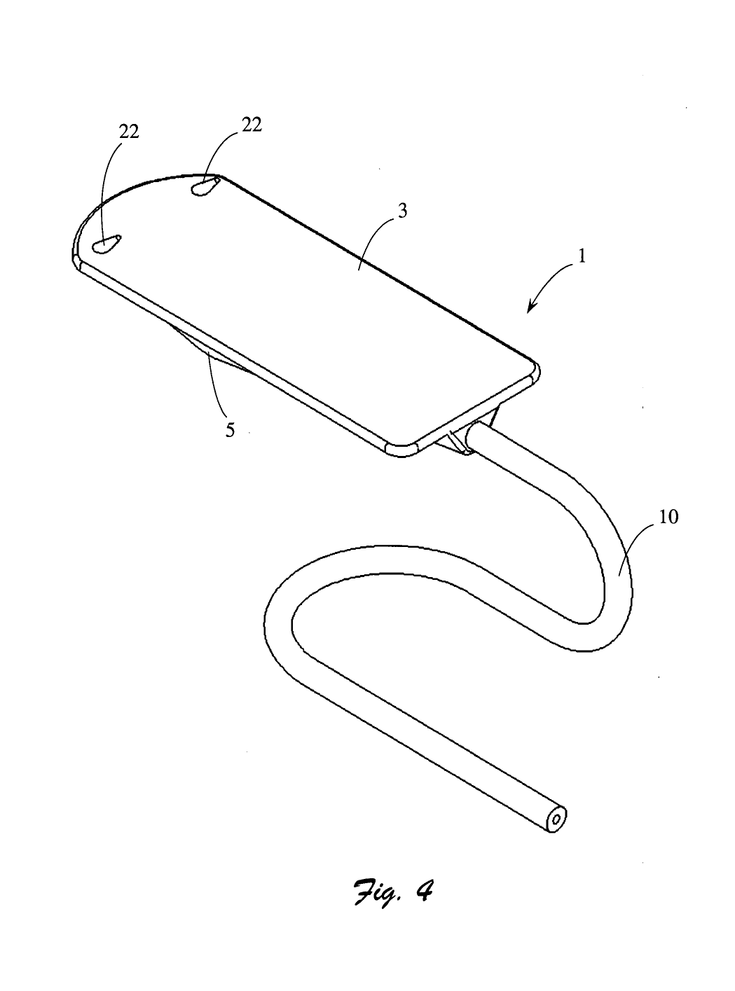

FIGS. 3 and 4 of the drawings show a pair of anti-migration barbs 22 which are spaced laterally from one another and project outwardly from the bottom of the foundation plate 3 of the sutureless venous access port 1. The pair of anti-migration barbs 22 are located at the front of foundation plate 3 and extend therefrom at an angle of about 45 degrees. However, the number and precise location of the barbs 22 may be changed without affecting the scope or advantages of this invention.

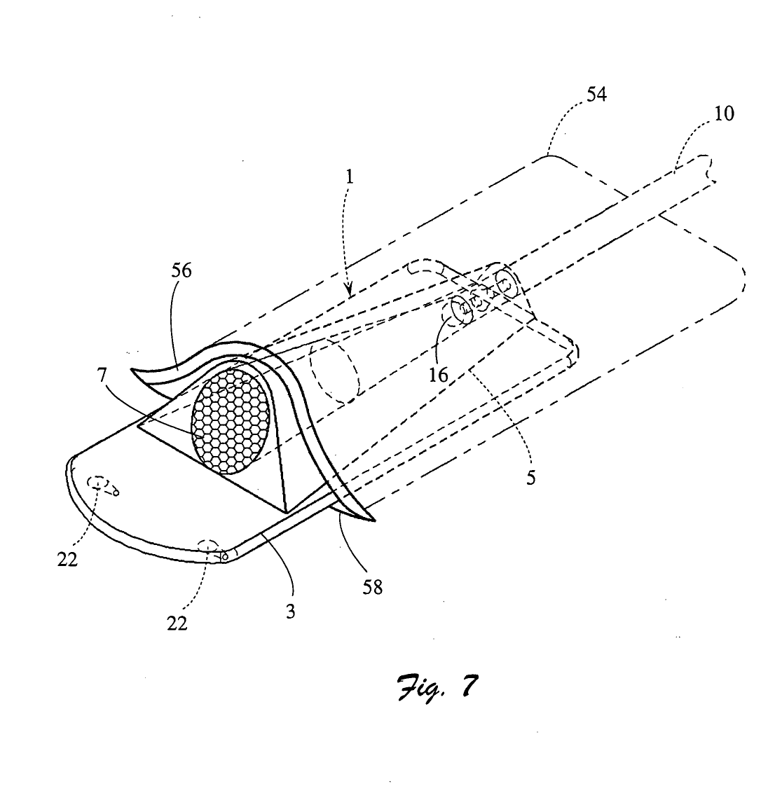

As an important improvement of the sutureless venous access port 1 herein disclosed, the anti-migration barbs 22 enable the hollow port body 5 to be attached to the patient's tissue without the requirement of sutures. Following implantation (and as will be described while referring to FIG. 7), the barbs 22 provide anchors to hold the port body 5 in place attached to the superficial muscular facia of the patient so as to prevent a migration or rotation thereof which might result in a kinking of the medication delivery lumen 10. Anti-migration barbs 22 advantageously avoid the necessity of sutures that are common to implanting the conventional (e.g., PORT-O-CATH) venous access port and the corresponding surgical procedure for attaching the conventional port to the patient's tissue.

Turning now to FIGS. 5A, 5B and 5C of the drawings, details of the spherical needle stop 16 are now disclosed. As previously indicated, a hollow lumen barb 18 projects from the narrow end of the needle puncture-resistant cone 12 that is surrounded by the hollow tapered body 5 of the sutureless venous access port 1. As also indicated, one end of the medication delivery lumen 10 is moved into surrounding engagement with the lumen barb 18 so that lumen 10 is coupled to the distal end of the hollow body 5 of access port 1 in fluid communication with cone 12. The spherical metal needle stop 16 is disposed at the narrow end of the puncture-resistant cone 12 ahead of the lumen barb 18 so as to lie against the engagement of the barb 18 and medication delivery lumen 10. In this regard, an inwardly projecting dimple or indentation 24 (best shown in FIG. 5C) is pressed into the narrow end of puncture-resistant cone 12. Dimple 24 is located so as to trap the needle stop 16 and prevent a displacement of the needle stop away from the narrow end of puncture-resistant cone 12.

As was also previously described, an incoming needle cannula 26 moving along the preferred needle target axis 9 (i.e., which makes an ideal angle of about 30 degrees with the horizontal plane of the foundation plate 3) will penetrate the self-sealing plug 7 for receipt at the interior 28 of the hollow puncture-resistant cone 12 by which the venous access port 1 is infused with fluid medication to be delivered to the circulatory system of a patient. The needle stop 16 is disposed against the narrow end of the puncture-resistant cone 12 to intercept and block the needle cannula 26 should it be pushed too far into the hollow body 5 and completely through the interior 28 of puncture-resistant cone 12. By virtue of the foregoing, the medication delivery lumen 10 will be shielded so as to avoid an accidental needle puncture and possible damage that might negatively affect the ability of lumen 10 to deliver medication from cone 12 to the patient.

Although the needle stop 16 is positioned at the narrow end of cone 12 to block the needle cannula 26 from moving therepast, the fluid medication with which the venous access port 1 is infused must be capable of flowing past needle stop 16 so as to be delivered to the patient via medication delivery lumen 10. To accomplish the foregoing, both the distal end of the hollow tapered port body 5 and the narrow end of the needle puncture-resistant cone 12 within which the needle stop 16 is located are distorted so as to have a non-round configuration (best shown in FIG. 5B). Because of this non-round configuration relative to the spherical configuration of needle stop 16, a fluid channel 30 is created between the distorted narrow end of puncture-resistant cone 12 and the opposite sides of needle stop 16 that are surrounded by cone 12. Accordingly, a fluid medication delivery path is established from needle cannula 16 through the venous access port 1 including the interior 28 of puncture-resistant cone 12, the fluid channel 30 around the sides of needle stop 16, and the medication delivery lumen 10, one end of which is coupled to lumen barb 18 in order to lie in fluid communication with fluid channel 30.

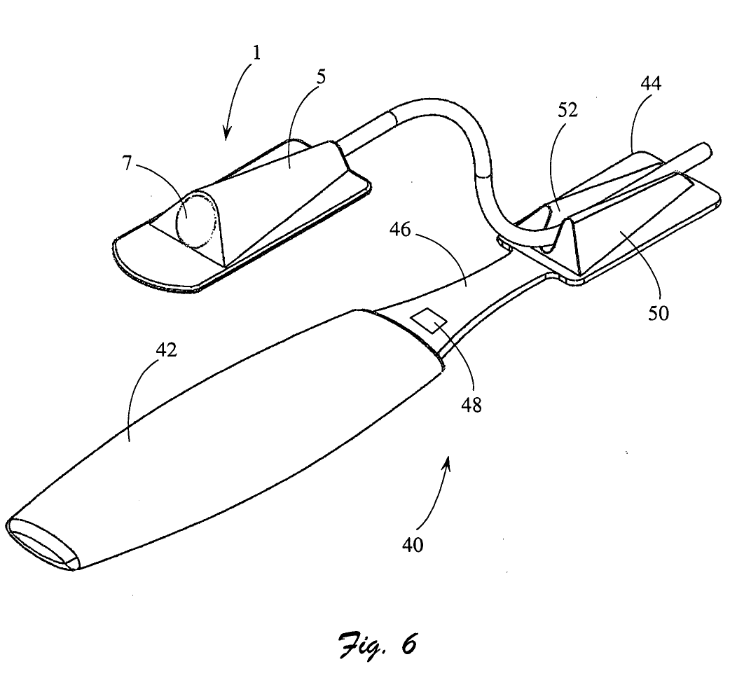

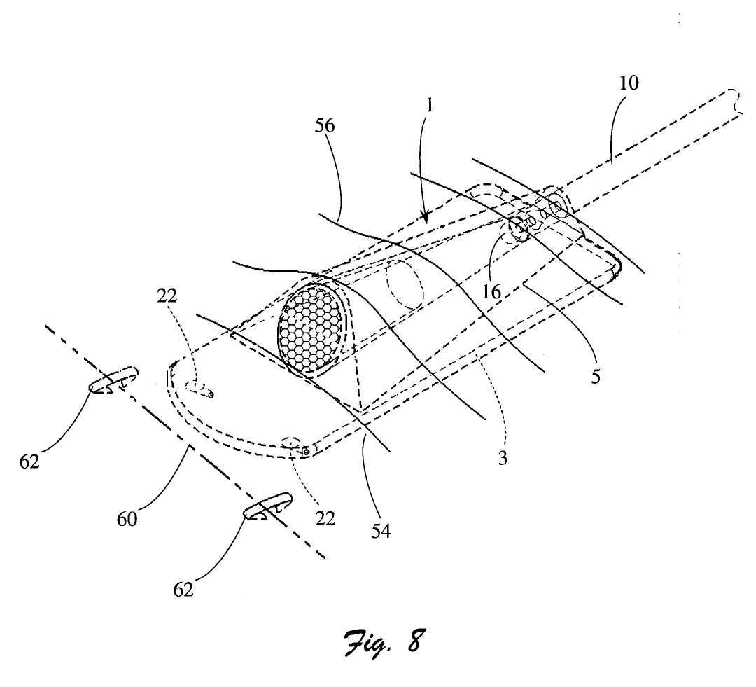

FIG. 6 of the drawings shows a pocket-forming tool 40 that is adapted to make a pocket (designated 54 in FIG. 8) below the patient's skin in which to receive the low profile sutureless venous access port 1 that has been described when referring to FIGS. 1-5. Pocket-forming tool 40 includes a handle 42 at one end, a pocket-forming head 44 at the opposite end, and a neck 46 extending therebetween. A soon-to-be-described position indicator 48 (e.g., a groove or a raised bump) is formed on the neck 46. Pocket-forming tool 40 is preferably manufactured from stainless steel or any other suitable autoclavable and reusable material.

The pocket-forming head 44 of pocket-forming tool 40 includes a tapered tissue dilator 50. A downwardly sloping lumen channel 52 runs axially along the tapered dilator 50. The lumen channel 52 is sized to receive therewithin and carry the medication delivery lumen 10 from access port 1. The dilator 50 helps to open an anti-migration pocket after the patient's skin has been incised and the pocket forming head 44 of tool 40 is moved inwardly of the incision.

As is best shown in FIG. 7 of the drawings, a small incision (designated 60 in FIG. 8) is initially made in the patient's skin with a scalpel, and the pocket-forming head 44 of the pocket-forming tool 40 of FIG. 6 is inserted through the incision to create a correspondingly small, anti-migration pocket 54 between a subcutaneous fat layer 56 an the muscularfacia 58 of a patient in need of regular fluid medication. The position indicator (designated 48 in FIG. 6) located on the neck 46 of tool 40 will line up with the incision when the pocket-forming head 44 has been sufficiently inserted between the patient's subcutaneous fat layer 56 and the muscularfacia 58 to create a pocket 54 that is accurately sized so as to be able to easily accommodate the venous access port 1, but small enough to avoid subjecting the patient to unnecessary trauma and recovery time.

Once the pocket 54 is formed, the pocket forming tool 40 is removed. The venous access port 1 is then pushed into the anti-migration pocket 54. The venous access port 1 should experience little resistance, because the pocket 54 has been pre-formed to the correct size. During implantation of the port 1, the medication delivery lumen 20 thereof is first cut to size and threaded through the pocket 54 to be attached to the (e.g., subclavian) vein of the patient by the earlier described Seldinger technique, or the like.

Once the venous access port 1 has been inserted within the anti-migration pocket 54, and referring to FIG. 8 of the drawings, the initial incision 60 is closed to allow healing. The foregoing may be accomplished by means of skin staples 62. Because only a small incision 60 is made, the requirement for more complicated sutures and the attendance of a surgeon may be advantageously avoided. After a sufficient healing time, the skin staples 62 are easily removed.

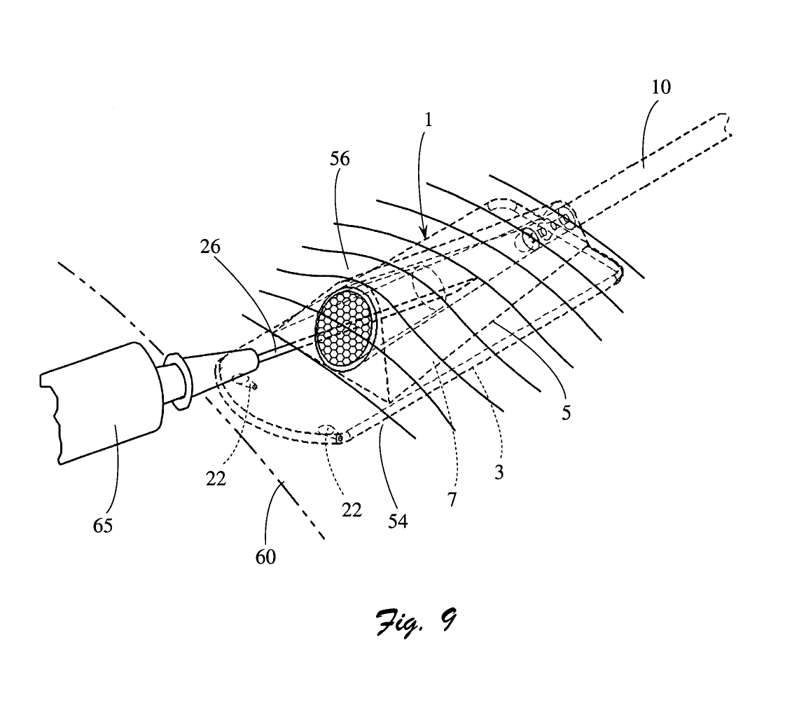

FIG. 9 of the drawings shows the anti-migration pocket 54 closed around the venous access port 1 to prevent a migration or rotation thereof. Because a small incision 60 is initially made in the patient's skin, the incision line may now be covered with a butterfly or similar small bandage (not shown). By virtue of the compact size of the low profile venous access port 1 herein disclosed, the patient's skin will not be stretched and will suffer little distortion. Therefore, the wound can heal relatively quickly compared to the healing time associated with implanting a conventional bulky subcutaneous venous access port. The accurate size of the preformed anti-migration pocket 54 and the anti-migration barbs 52 projecting from the foundation plate 3 of access port 1 for attachment to the patient's muscular facia cooperate to resist a movement of the port and a possible kinking of the medication delivery lumen 10.

When the patient's wound has healed, a syringe 65 may be moved into fluid communication with the venous access port 1 as often as necessary via a needle cannula 26 and the self-healing gelatinous plug 7 of port 1 (previously described when referring to FIG. 5A). Thus, a measured supply of fluid medication with which the access port 1 is infused can be delivered directly to the patient's circulatory system on an ongoing basis by way of medication delivery lumen 10. Because the cannula 26 penetrates the target area of port 1 at a small acute angle (designated 8 in FIG. 1) relative to the patient's skin rather than a 90 degree angle as is otherwise common to many conventional ports, there is less likelihood that the cannula might inadvertently puncture the delivery lumen 10 as a consequence of a misalignment with the port.

By virtue of the sutureless venous access port 1 of the present invention and the pocket forming tool 40 for creating a subcutaneous anti-migration pocket within which port 1 is inserted and surrounded, the need for a surgical procedure, the cost associated with an operating room, and the trauma and recovery time suffered by the patient are all advantageously reduced or avoided altogether. Thus, the procedure for implanting the venous access port 1 can be performed in a radiology lab by a radiologist on an outpatient basis instead of a surgeon working in an operating theater. Moreover, because of its compact and streamlined size (relative to the conventional PORT-O-CATH port), the venous access port 1 described herein will be more comfortable to the patient and cosmetically less obtrusive.

1. A fluid port to be implanted below the skin of a patient and infused with a liquid supplied by a needle cannula so that the liquid can be delivered to the circulatory system of the patient, said fluid port including a hollow port body within which to receive the liquid from the needle cannula, a fluid delivery lumen coupled to said hollow port body so that the liquid with which said fluid port is infused is delivered to the patient's circulatory system, and an attachment adapted to hold said fluid port against the tissue of the patient so as to prevent a displacement thereof without the use of sutures.

2. The fluid port recited in claim 1, wherein said attachment is at least one barb projecting from said fluid port by which to hold said fluid port against the tissue of the patient.

3. The fluid port recited in claim 2, further including a base to be affixed to the patient's tissue, said at least one barb projecting from said base by which to anchor said fluid port against the tissue of the patient.

4. The fluid port recited in claim 3, further including a hollow infusion chamber located inwardly of and surrounded by said hollow port body so as to receive the liquid supplied by the needle cannula, said fluid delivery lumen lying in fluid communication with said hollow infusion chamber, said hollow infusion chamber being manufactured from a needle puncture-resistant material.

5. The fluid port recited in claim 4, wherein said hollow infusion chamber has a fluid inlet end, a fluid outlet end, and a lumen connector at said fluid outlet end, said fluid delivery lumen mated to the lumen connector at said fluid outlet end so that said fluid delivery lumen lies in said fluid communication with said infusion chamber.

6. The fluid port recited in claim 5, further including a needle stop located within said hollow infusion chamber at said fluid outlet end thereof to intercept and prevent a needle cannula from penetrating said lumen connector and piercing said fluid delivery lumen mated thereto.

7. The fluid port recited in claim 6, wherein said needle stop has a spherical shape and the fluid outlet end of said hollow infusion chamber has a non-circular shape, such that a fluid channel is established between said spherical needle stop and said non-circular fluid outlet end to enable the said fluid communication between said hollow infusion chamber and said fluid delivery lumen.

8. The fluid port recited in claim 7, wherein said hollow infusion chamber has a conical configuration, whereby said fluid inlet end thereof is relatively wide and said fluid outlet end is relatively narrow, said narrow fluid outlet end being deformed to have said non-circular shape, and said spherical needle stop being located at said relatively narrow fluid outlet end in axial alignment with said fluid delivery lumen.

9. The fluid port recited in claim 4, wherein said hollow infusion chamber has a fluid inlet end, a fluid outlet end, a longitudinal axis extending between said fluid inlet and outlet ends, and a self-sealing plug located at said fluid inlet end for penetration by the needle cannula when said fluid port is infused with liquid.

10. The fluid port recited in claim 9, further including a target axis axially aligned with the self-sealing plug located at the fluid inlet end of said hollow infusion chamber and the longitudinal axis of said infusion chamber, said target axis and the skin of the patient below which said fluid port is implanted forming an angle of less than 90 degrees.

11. The fluid port recited in claim 10, wherein the angle formed by said target axis and the patient's skin is an acute angle between 20 and 45 degrees.

12. The fluid port recited in claim 10, further including a needle stop located within said hollow infusion chamber at the fluid outlet end thereof, said needle stop being axially aligned with said target axis, said self-sealing plug, and the longitudinal axis of said hollow infusion chamber.

13. A combination, comprising:
a fluid port to be implanted below the skin of a patient and infused with a liquid supplied by a needle cannula so that the liquid can be delivered to the circulatory system of the patient, said fluid port including a hollow needle puncture-resistant infusion chamber to receive the needle cannula and the liquid supplied thereby, a fluid delivery lumen lying in fluid communication with said infusion chamber so that the fluid received by said infusion chamber is delivered to the patient's circulatory system, and at least one barb by which said fluid port is attached to the tissue of the patient to prevent a displacement thereof without the use of sutures; and
a pocket forming tool to move through an incision made in the patient's skin to form an anti-migration pocket in which to receive and surround said fluid port.

14. The combination recited in claim 13, wherein the needle puncture-resistant infusion chamber of said fluid port is a cone having a relatively wide fluid inlet end, a relatively narrow fluid outlet end, a self-sealing plug located at said fluid inlet end, and a needle stop located at said fluid outlet end, said fluid delivery lumen coupled to the fluid outlet end of said infusion chamber.

15. The combination recited in claim 14, wherein the conical needle puncture-resistant infusion chamber of said fluid port has a longitudinal axis extending between the fluid inlet and outlet ends thereof, said longitudinal axis and the skin of the patient below which said fluid port is implanted forming an angle of less than 90 degrees.

16. The combination recited in claim 13, wherein said pocket forming tool has a pocket forming head at one end thereof to be inserted through the incision in the patient's skin to form said anti-migration pocket, said pocket forming head and said fluid port having a substantially identical size so that said fluid port can be received within said anti-migration pocket.

17. The combination recited in claim 16, wherein said pocket forming tool includes a position indicator to be aligned with the incision in the patient's skin and thereby indicate the insertion of said pocket forming head through the incision to form said anti-migration pocket.
